(19) United States
(12) Patent Application Publication
(10) Pub. No.: US 2024/0087706 A1
(43) Pub. Date: Mar. 14, 2024

(54) COMPUTER-IMPLEMENTED METHOD AND SYSTEM FOR PROVIDING PERSONALIZED MEDICAL SUPPLIES TO A PATIENT

(71) Applicant: 9451-0138 Quebec inc., Quebec (CA)

(72) Inventor: Martin Gladu, Quebec (CA)

(21) Appl. No.: 18/463,810

(22) Filed: Sep. 8, 2023

Related U.S. Application Data
(60) Provisional application No. 63/374,983, filed on Sep. 8, 2022.

(51) Int. Cl. G16H 20/10; G06F 16/951; G06F 21/31; G06Q 30/0601

(52) U.S. Cl. CPC G16H 20/10; G06F 16/951; G06F 21/31; G06Q 30/0603; G06Q 30/0613; G06Q 30/0635; G06Q 30/0643

(57) ABSTRACT

A system to automatically provide personalized medical supplies to a patient generally aims at gathering patient information, such as but not limited to anatomical dimensions, prescriptions, medical conditions and any other parameters impacting the use of a medical supply is provided. The system generally uses such information to filter or generate a personalized subset of medical supplies fulfilling the medical prescription while having properties or attributes matching or being within an acceptable range of information relating to the patient. The patient may then buy one or more medical supplies within the subset presented to the patient thereof.

### CROSS-REFERENCE TO RELATED APPLICATIONS

The present patent application claims the benefits of priority of U.S. Provisional Patent Application No. 63/374,983, entitled “COMPUTER-IMPLEMENTED METHOD AND SYSTEM FOR PROVIDING PERSONALIZED MEDICAL SUPPLIES TO A PATIENT” and filed at the United States Patent and Trademark Office on Sep. 8, 2022, the content of which is incorporated herein by reference.

### FIELD OF THE INVENTION

The present invention generally relates to computer systems and methods to provision medical supplies to a patient. More specifically, the present invention relates to computer systems and methods to provision medical supplies to a patient based on the medical conditions or on anatomic parameters of the patient.

### BACKGROUND OF THE INVENTION

Some medical supplies are tightly linked to the morphology or to the medical condition of a patient. As such, the selection of the medical supply fitting the patient involves using the parameters related to the said morphology or medical condition of the patient to identify the right product in a catalogue of a medical supplier or in multiple catalogues associates with a plurality of suppliers. One example of medical supplies which require such selection are compression socks.

Healthcare professionals, such as nurses or medical technician, are typically performing the assessment of the medical condition and morphology of the patient and are identifying some medical supplies (i.e., compression socks) which may be used to fulfill a medical prescription. Such tasks involve looking at a plurality of catalogues to select the supplies matching the parameters associated with the condition of the patient. The selection of a subset of medical supplies requires important efforts and the healthcare professional may overlook a specific product or may include a product with an inadequate fit for the patient.

Upon presenting the subset of products to the patient, the said patient may then select the product having the preferred esthetics or designs.

The drawbacks of the present method of selecting a fitting product notably include using precious time of the healthcare professional, which time could be used to performed other medical tasks for other patients, increased risks of errors by the healthcare professional, limited choices for the patient, etc. There is thus a need for an improved method and system for providing personalized medical supplies to a patient overcoming the drawbacks of the prior art.

### SUMMARY OF THE INVENTION

The shortcomings of the prior art are generally mitigated by a computer-implemented method for providing personalized medical supplies to a patient based on medical condition and measurements of the patient. The method comprises inputting patient data into a server, the patient data comprising prescription data associated with the medical condition of the patient and anatomical data in relation with one or more body portion relating to the prescription data, the inputting of the prescription data triggering automatic matching of a plurality of medical supplies from a data source of medical supplies fitting the medical condition of the patient associated with the inputted prescription data of the patient, generating and storing a filter to associate the matching medical supplies to the patient data and communicating to a computerized device of the patient only the medical supplies associated with the filter upon receiving a request from the said computerized device of the patient.

The method may further comprise fetching from a plurality of third-party systems data relating to third-party medical suppliers, the fetching of the data of each of the third-party system triggering automatic conversion of the third-party medical suppliers data into a predetermined format and storing the converted data in the data source of medical supplies.

The computer-implemented method of claim 3, the conversion further comprising mapping a plurality of attributes of the third-party medical supplies to a respective attribute of the predetermined format.

The inputting of the prescription data may comprise inputting a date of a prescription, a disease, an anatomical region or portions of the body being treated, dimensions of the anatomical region and treatment indications. The input of the prescription data may be performed by a healthcare professional.

The computer-implemented method may further comprise the patient selecting one or more of the communicated medical supplies of the filter and ordering the said selected medical supplies. The computer-implemented method may further comprise linking the medical data of a plurality of patients in relation to medical supplies ordered by the said patients.

The computer-implemented method may further comprise a healthcare professional adding or removing one or more medical supplies to the generated filter associated with the patient data. The automatic matching of a plurality of medical supplies from a data source of medical supplies may further comprise matching medical supplies only fitting stored patient anatomical dimensions and fulfilling the prescription data of the patient.

The computer-implemented method may further comprise sending an electronic message to a computerized device of the patient prior to a predetermined duration before the selected medical supply of the patient has to be renewed to ensure an adequate treatment.

The medical supplies may be compression socks.

In another aspect of the invention, a system for providing personalized medical supplies to a patient based on medical condition and measurements of the patient is provided. The system comprises a login module to authenticate a user to the system, a third-party medical supplies module configured to store a plurality of medical supplies from third-parties into a predetermined format a patient module and a healthcare professional module. The patient module comprises a data store comprising data relating to patients, the data comprising anatomical data of the patient and prescription data, a medical supplies matcher configured to match a plurality of medical supplies of the third-party medical supplies module fitting the anatomical data of the patient and fulfilling the inputted prescription data of the patient and to generate and store a filter to associate the matching medical supplies to the patient data. The patient module further comprises a catalogue browser configured to display only the medical supplies associated with the filter and an order module for a user to select and add to a list of desired products only the one or more products associated with one of the generated filters associated with the patient data and displayed by the catalogue browser. The healthcare professional module comprises a data store comprising a list of patients associated with a healthcare professional, an interface to input prescription data associated to one or more of the patients of the list and an interface to input or capture measurements of anatomical regions of one or more of the patients of the list.

The healthcare professional module may further comprise a catalogue browser simulator for simulating the catalogue browser of the patient module of one of the patients of the list.

The system may further comprise an importation module for automatically retrieving and storing third-party medical supplies data from a plurality of third-party systems into the third-party medical supplies module. The importation module may further comprise a plurality of data connectors, each data connector being configured to establish communication with a third-party system and to transfer data from the said third-party system. The system may further comprise a plurality of medical data converters, each data converter being configured to transform the retrieved third-party medical supplies data from a third-party specific format into a predetermined format usable by the system. The converters may be configured to map a plurality of attributes of the third-party medical supplies to a respective attribute of the predetermined format.

The system may further comprise a crawler for analyzing potential relationships between characteristics of patient users and medical supplies that are ordered by such patients. The crawler may use a trained artificial intelligence module to perform the analysis.

The medical supplies may be compression socks.

Other and further aspects and advantages of the present invention will be obvious upon an understanding of the illustrative embodiments about to be described or will be indicated in the appended claims, and various advantages not referred to herein will occur to one skilled in the art upon employment of the invention in practice.

### BRIEF DESCRIPTION OF THE DRAWINGS

The above and other aspects, features and advantages of the invention will become more readily apparent from the following description, reference being made to the accompanying drawings in which:

FIG. 1 is a workflow diagram of an embodiment of a computer-implemented method and system for providing personalized medical supplies to a patient in accordance with the principles of the present invention.

FIG. 2 is an illustration of an exemplary interface listing the patients of a system for providing personalized medical supplies to a patient in accordance with the principles of the present invention.

FIG. 3 is an illustration of an exemplary interface displaying information related to a patient of a system for providing personalized medical supplies to a patient in accordance with the principles of the present invention.

FIG. 4 is an illustration of an exemplary interface displaying a subset of compression socks fitting a specific patient of a system for providing personalized medical supplies to a patient in accordance with the principles of the present invention.

FIG. 5 is an illustration of an exemplary interface allowing sending fitting compression socks to a patient of a system for providing personalized medical supplies to a patient in accordance with the principles of the present invention.

FIG. 6 is an illustration of an exemplary interface for editing a prescription for a specific patient of a system for providing personalized medical supplies to a patient in accordance with the principles of the present invention.

### DETAILED DESCRIPTION OF THE INVENTION

A novel computer-implemented method and system for providing personalized medical supplies to a patient will be described hereinafter. Although the invention is described in terms of specific illustrative embodiments, it is to be understood that the embodiments described herein are by way of example only and that the scope of the invention is not intended to be limited thereby.

The method and system for providing personalized medical supplies to a patient is generally aimed at patients receiving home medical treatments, having diseases or medical conditions limiting their movement, living outside of cities or near stores selling medical supplies or patients preferring a simple method of ordering and receiving medical supplies. The said method and system generally aim at getting access to a product or medical supply meeting the medical conditions of the patient amongst thousands of SKUs relating to the medical supplies.

Broadly, the healthcare professional identifies the specific needs of the patient according to the medical conditions or needs of the patient. Such needs or conditions are inputted in the system which identifies the products or supplies matching the specific needs of the medical condition of the patient amongst thousands of products available from a plurality of suppliers. The system generates a catalogue of products matching the needs of the patient, the catalogue being personalized to the specific needs of the patient.

The patient, through a computerized device, selects the products that meets his/her personal needs or preferences (such as budget considerations, preferred colors or style, brand, etc.) amongst products matching their specific medical condition.

As such, the system aims at ensuring that the patient order only products matching his/her specific medical needs. The patient further gains access to a large selection of products, regardless of the location of the patient. As such, the level of success of the treatment is typically increased while the patient may stay at home or in his/her actual residence.

The system to automatically provide personalized medical supplies to a patient generally aims at gathering patient information, such as but not limited to anatomical dimensions, prescriptions, medical conditions and any other parameters impacting the use of a medical supply. The system uses such information to filter or generate a personalized subset of medical supplies fulfilling the medical prescription while having properties or attributes matching or being within an acceptable range of information relating to the patient.

The system further aims at providing a first interface for a healthcare professional to create and update a medical record of a patient and to create a catalogue of a subset of medical supplies personalized to the patient based on the specific data of the medical record, such as the prescription data and measurements. The subset of products is selected from a plurality of suppliers each having at least thousands of variants of products. As such, the subset data substantially limits the risk factors associating with a healthcare professional selecting a product not fitted for the medical condition of a patient. Indeed, selecting the wrong compression socks for a patient may create other medical conditions which may, in some cases, be worse than the initial medical condition. The system further provides a second and distinct interface for a patient to select a medical supply. The said second interface generally aims at a patient to select and buy the products based on subjective parameters such as esthetic, comfort, colors, etc. As such, the patient may only buy products which are adequate for its medical condition.

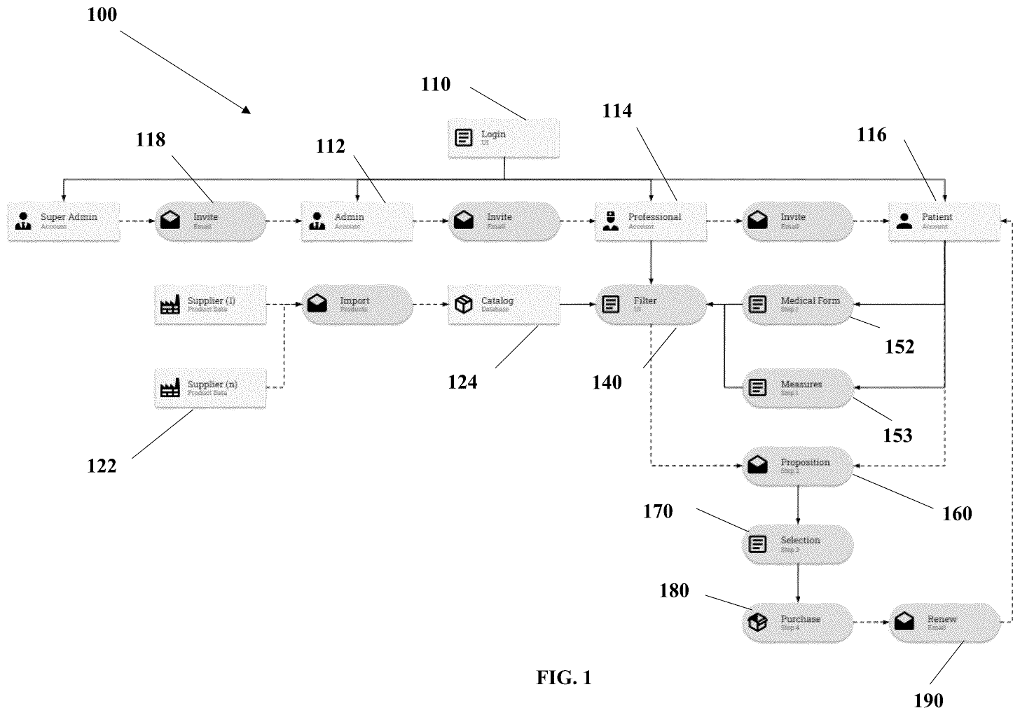

Referring now to FIG. 1, a workflow diagram of an embodiment of a method and system for providing personalized medical supplies to a patient is illustrated. The system 100 generally comprises a login module 110 configured to give access to a user to the system. Understandably, based on the type of user, the login module 110 may allow a different access to the system 100. In one embodiment, the system 100 may comprise administrator users 112, healthcare professional users 114 and patient accounts 116.

The system 100 may be configured to send to a healthcare professional or a patient one or more invites through an electronic message 118, such as an email or an SMS, or through any other method of inviting a user. Upon registering with the system 100, the invited user 114 or 116 gains access to the system 100. The access associated with the user 114 or 116 is function of the type of user.

Still referring to FIG. 1, the system may comprise a module for fetching and storing data relating to product data 120 of a plurality of suppliers 122. The module may be also referred as the importation module. The said module 120 typically accesses the data of suppliers through any type of data connector, such as, but not limited to, an application programming interface (API), a web service or retrieving data bundled as a file or a plurality of files. The retrieved data of the suppliers may be stored in a data source 124, also referred as the catalog.

The system may further comprise a supplier data converter module. The supplier data converter module generally comprises a plurality of converters for converting the data from the supplier format into a format usable by the system. Each converter maps an attribute of a product or supply of the supplier to a respective attribute of the format usable by the system. The converted data is stored in a data source, such as a database system. As such, the system may comprise a map table specific to each supplier or to each type of data of a supplier. When the data converter module accesses or retrieves an attribute of a product, the system tries to identify a matching attribute in the map table.

In some embodiments, the modules for fetching supplier data and for converting said fetched data to a format usable by the system may be executed at predetermined frequency or each time a supplier changes data relating to the products. Understandably, any known method to synchronize and update the data of the suppliers in the data source of the system may be used within the scope of the present invention.

The system may further comprise a healthcare professional module or interface. As such, when the system identifies the current user as being a healthcare professional user, the system displays the healthcare professional interface. The said interface generally comprises a list of patients associates with the healthcare professional, a catalogue browser for simulating the catalogue accessible for a patient selected from the said list of patients, an interface to input prescription data associated to a specific user and an interface to input or capture measurements of one the patients of the said list. In some embodiments, the healthcare professional interface allows the healthcare professional to enter measurements taken on a patient, to enter any pending prescription of the said patient and to generate the filter to display the catalogue personalized for the patient.

The system may be implemented using a web server, a database server and computerized devices having access to the web server through a network, such as the Internet. The web server is in data communication with third-party systems through the network, allowing retrieval of the products from a plurality of suppliers. The computerized device may be configured to run a software or to connect to the web server through a browser. The web server may be configured to execute a software or a program providing a graphical user interface to the patient user, to the healthcare professional user or to other external users. The above described modules may be configured to be executed on a computer, such as the web server.

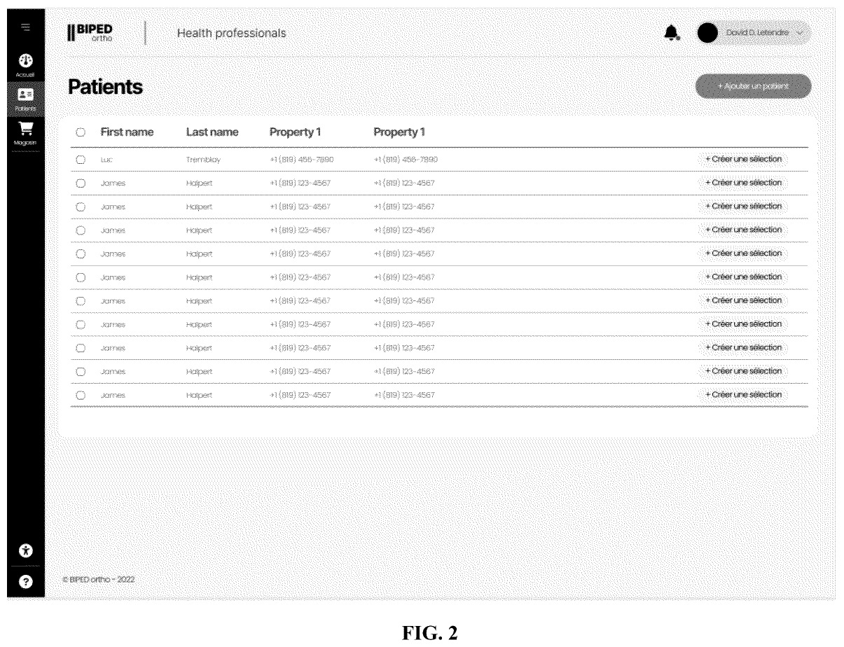

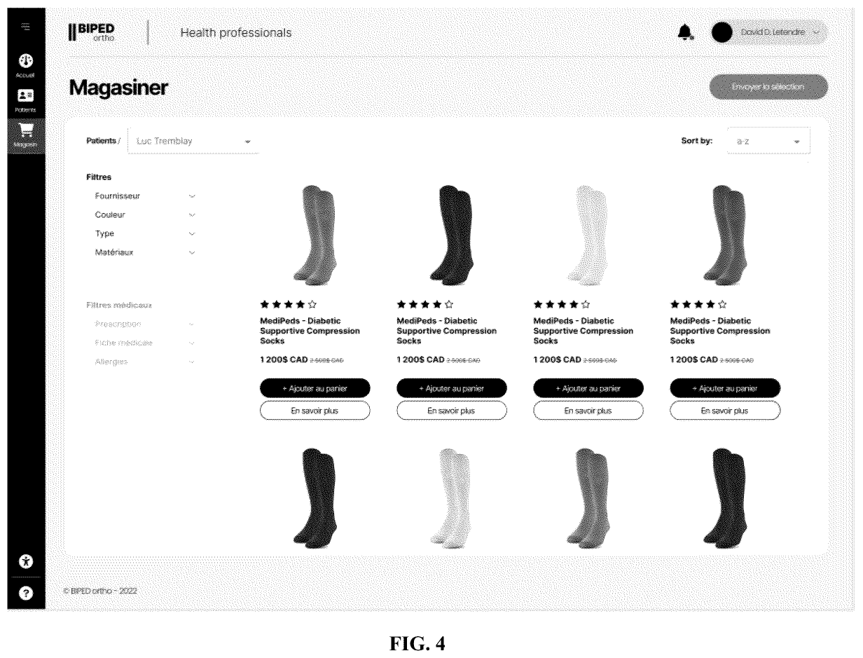

Referring to FIG. 2, an exemplary graphical user interface showing a list of patients of the system is illustrated. The system further comprises patient module which generally holds data relating to the patients using the system or to patients receiving treatments from the healthcare professionals using the system. The patient module comprises records comprising identification information of the patient, such as but not limited to, the name, birth date, address or any other relevant identification data. The patient module further comprises prescription data associated with the patient. The prescription data may comprise the date of the prescription, the disease, anatomical region or portions of the body being treated, and the treatment indications, such as medications type or quantity or medical supply to be fitted. In an example, the prescription could comprise treating a tibia with a sock having a compression of 20-40 mmHg, as shown at FIG. 4. The prescription data may comprise more than one prescription for each patient.

Still referring to FIG. 2, the graphical user interface comprises a function to create a selection for a selected patient in the list.

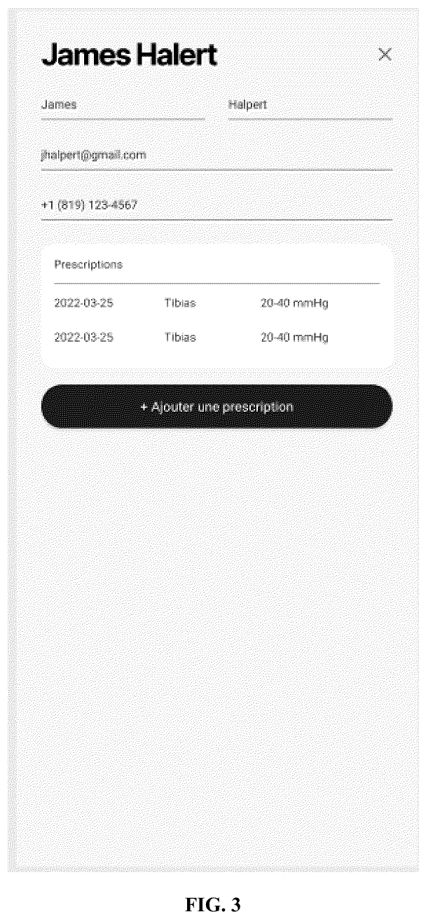

Referring now to FIG. 3, an exemplary graphical user interface showing prescriptions associated to a patient of the system is illustrated. The said graphical user interface generally allows display of the prescription data associated to a patient, as described above.

Referring now to FIG. 4, an exemplary graphical user interface showing a store or subset of products fitting the patient dimensions and fulfilling one or more prescription of the patient is illustrated. The said graphical user interface may be displayed when the system is used by the health care professional. As such, the health care professional may proceed to buy the medical supply, such as compression socks, for a specific patient or may display the said subset of medical supplies to a patient. As discussed above, the system displays only the adequate medical supplies for the patient, thus aiming at minimizing the risk of errors by the health care professional.

The patient module further comprises records about dimensions of anatomical regions or body regions of the patient taken by the heath professional during a medical visit or a consultation with the patient.

The system is configured that only a healthcare professional user modifies a patient record, such as adding any prescription data or dimensions of anatomical regions or body regions of the patient which were taken by the healthcare professional or to update any type of medical data of the patient. As such, in a preferred embodiment, the patient user may not be authorized to modify or complete any data associated with its medical record, such as prescription data or measurements.

Referring back to FIG. 1, the system 100 is further configured to allow a healthcare professional to request the system to generate or create a subset catalogue for a patient in regard to one or more prescription present in the patient record 140, the data associated to a medical follow-up on the patient of the healthcare professional, such as the data of the filled medical form 152, and/or the measurements taken on the patient by the healthcare professional. As such, the system creates a filter object 140 configured to select only the products from the catalogue of multiple suppliers which fit the conditions of the patient and which fulfill the one or more prescriptions associated to the patient. The generated filter 140 is typically saved and stored in a data source of the system and associated to the patient. In some embodiments, such as embodiments offering different categories of medical supplies (i.e., compression socks and diapers), the generated filter 140 may be associated or linked to one or more prescriptions or to specific conditions within a specific category of medical supplies. As an example, a first filter may be stored for a subset of compression socks based on the compression socks prescriptions of the patient and a second filter may be stored for a subset of diapers for the same patient based on measurements. Understandably, any number of filters may be created for a patient.

Still referring to FIG. 1, the system 100 comprises a patient data module 150. The patient data module 150 may comprise a medical form 152 to be filed by the by the healthcare professional user 114. The medical form 152 may require inputting medical information relating to the patient, such as but not limited to, medical condition, diseases, medication taken by the patient, surgeries, etc.

The system 100 further comprises a measurement module 153. The measurement module 152 allows a user, such as the patient user 116 or the healthcare professional user 114, to enter latest measurements of anatomical regions or body portions of the patient. In some embodiments, the entered measurements may be associated to a date where the said measurement where physically taken on the patient.

The system 100 further comprises a proposition module 160. The proposition module 160 uses the filter 140 generated by the system 100 to present products from a plurality of suppliers fitting the measurements of the patient and fulfilling the prescription or medical condition of the patient. The proposition 160 may be displayed to the user 114 or 116 as a store using well known filters or sort functions.

In some embodiments, the proposition 160 generated by the system 100 may be modified by a healthcare professional user 114. As such, the healthcare professional user 114 may add other products or remove products from the subset based on medical conditions observed by the healthcare professional user 114.

The system 100 further comprises a selection module 170. The selection module 170 allows a user 114, 116 to select and add to a list of desired products one or more products from the customized product list displayed by the proposition module 160.

The system 100 further comprises a purchase module 180. The purchase module 180 is configured to allow a user to buy the products present in the selection 170. Understandably, any known method allowing buying a set of products over a network, such as the internet, may be used within the scope of the present invention. As such, the purchase module 180 may contact an external payment processor to conclude the transaction over a network.

The system 100 may further comprise a renewal module 190. The renewal module 190 is configured to send an electronic message to a user 114, 116 prior to a predetermined duration before a medical supply of the patient has to be renewed to ensure a good treatment. As such, the renewal module 190 may be configured to send an email to a user 114, 116 three (3) months prior to the typical end-of-life of medical supply bought by the user.

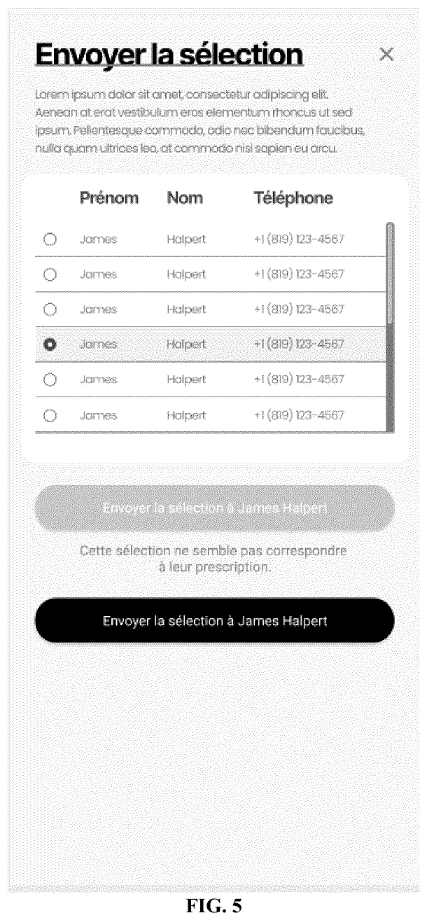

Referring to FIG. 5, an embodiment of a graphical user interface for sending the subset of products resulting of the filter is illustrated. In such embodiment, the system displays an interface accessible by the healthcare professional comprising contact information of the patient. Once the correct contact information is selected, the system may check that the associated subset of products fit the selected patient and an fulfill the prescription.

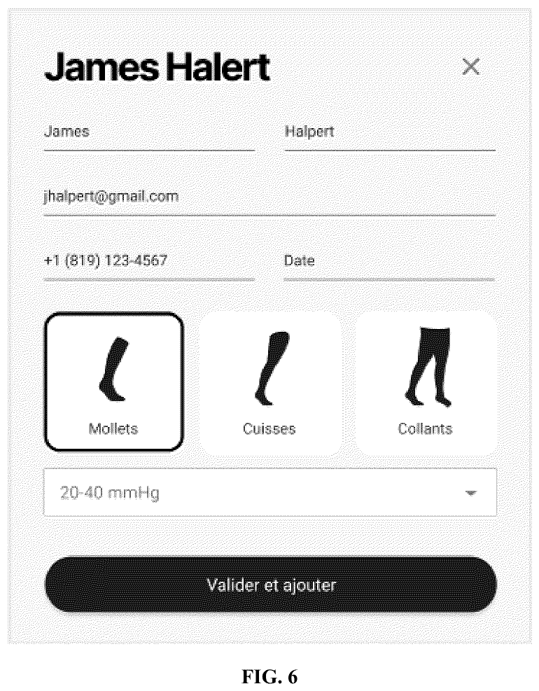

Referring now to FIG. 6, an embodiment of a graphical user interface for viewing or editing a prescription is illustrated. In some embodiments, the healthcare professional is authorized to add or edit one or more prescriptions of a patient user 116. As such, the prescription data may comprise the type of medical supply (i.e., knee-high socks, pantyhose, thigh-high, etc.), the date of the prescription, the name of physician or doctor, the identification number of the license of the physician or doctor, the name of the patient, any specific dose or parameters (i.e., tightness of the compression sock). In some embodiments, a photo of the original prescription is uploaded and associated with the prescription data.

In some other embodiments, the system 100 may be further configured to link the medical data of a plurality of patients in relation to products bought by the said patients. In such embodiments, the system 100 may comprise a crawler or a process analyzing potential relationships between characteristics of patient users 116, such as medical conditions, measurements, etc., and the products that are ultimately bought by such patients 116.

In yet other embodiments, the analyzing process may be replaced with an artificial intelligence module being trained with the data from the transactions and the medical data of the users.

In some further embodiments, the system 100 may comprise a feedback module to be accessed by the healthcare professional 114 or patient 116 users. The system 100 may be configured to determine if a product or a plurality of product are well-received or work as expected for the patient users 116. The system 100 may be configured to collect and store the feedback information over time to further identifies a trend or an evolution of the perception of the users 116. The feedback module may create link between the feedback information and the sales of the different products.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

1. A computer-implemented method for providing personalized medical supplies to a patient based on medical condition and measurements of the patient, the method comprising:
inputting patient data into a server, the patient data comprising prescription data associated with the medical condition of the patient and anatomical data in relation with one or more body portion relating to the prescription data;
the inputting of the prescription data triggering automatic matching of a plurality of medical supplies from a data source of medical supplies fitting the medical condition of the patient associated with the inputted prescription data of the patient;
generating and storing a filter to associate the matching medical supplies to the patient data;
communicating to a computerized device of the patient only the medical supplies associated with the filter upon receiving a request from the said computerized device of the patient.

2. The computer-implemented method of claim 1, further comprising
fetching from a plurality of third-party systems data relating to third-party medical suppliers;
the fetching of the data of each of the third-party system triggering automatic conversion of the third-party medical suppliers data into a predetermined format; and
storing the converted data in the data source of medical supplies.

3. The computer-implemented method of claim 2, the conversion further comprising mapping a plurality of attributes of the third-party medical supplies to a respective attribute of the predetermined format.

4. The computer-implemented method of claim 1, inputting the prescription data comprising inputting a date of a prescription, a disease, an anatomical region or portions of the body being treated, dimensions of the anatomical region and treatment indications.

5. The computer-implemented method of claim 1, the input of the prescription data being performed by a healthcare professional.

6. The computer-implemented method of claim 1 further comprising the patient selecting one or more of the communicated medical supplies of the filter and ordering the said selected medical supplies.

7. The computer-implemented method of claim 6 further comprising linking the medical data of a plurality of patients in relation to medical supplies ordered by the said patients.

8. The computer-implemented method of claim 1 further comprising a healthcare professional adding or removing one or more medical supplies to the generated filter associated with the patient data.

9. The computer-implement method of claim 1, the automatic matching of a plurality of medical supplies from a data source of medical supplies further comprising matching medical supplies only fitting stored patient anatomical dimensions and fulfilling the prescription data of the patient.

10. The computer-implemented method of claim 1 further comprising sending an electronic message to a computerized device of the patient prior to a predetermined duration before the selected medical supply of the patient has to be renewed to ensure an adequate treatment.

11. The computer-implemented of claim 1, the medical supplies being compression socks.

12. A system for providing personalized medical supplies to a patient based on medical condition and measurements of the patient, the system comprising:
a login module to authenticate a user to the system;
a third-party medical supplies module configured to store a plurality of medical supplies from third-parties into a predetermined format;
a patient module comprising:
a data store comprising data relating to patients, the data comprising anatomical data of the patient and prescription data;
a medical supplies matcher configured to:
match a plurality of medical supplies of the third-party medical supplies module fitting the anatomical data of the patient and fulfilling the inputted prescription data of the patient;
generate and store a filter to associate the matching medical supplies to the patient data;
a catalogue browser configured to display only the medical supplies associated with the filter;
an order module for a user to select and add to a list of desired products only the one or more products associated with one of the generated filters associated with the patient data and displayed by the catalogue browser;
a healthcare professional module comprising:
a data store comprising a list of patients associated with a healthcare professional;
an interface to input prescription data associated to one or more of the patients of the list; and
an interface to input or capture measurements of anatomical regions of one or more of the patients of the list.

13. The system of claim 12, the healthcare professional module further comprising a catalogue browser simulator for simulating the catalogue browser of the patient module of one of the patients of the list.

14. The system of claim 12 further comprising an importation module for automatically retrieving and storing third-party medical supplies data from a plurality of third-party systems into the third-party medical supplies module.

15. The system of claim 14, the importation module further comprising a plurality of data connectors, each data connector being configured to establish communication with a third-party system and to transfer data from the said third-party system.

16. The system of claim 14 further comprising a plurality of medical data converters, each data converter being configured to transform the retrieved third-party medical supplies data from a third-party specific format into a predetermined format usable by the system.

17. The system of claim 16, the converters being configured to map a plurality of attributes of the third-party medical supplies to a respective attribute of the predetermined format.

18. The system of claim 12 further comprising a crawler for analyzing potential relationships between characteristics of patient users and medical supplies that are ordered by such patients.

19. The system of claim 18, the crawler using a trained artificial intelligence module to perform the analysis.

20. The system of claim 1, the medical supplies being compression socks.
